(19) United States
(12) Patent Application Publication
(10) Pub. No.: US 2007/0270925 A1
(43) Pub. Date: Nov. 22, 2007

(54) Method and apparatus for non-invasively removing heat from subcutaneous lipid-rich cells including a coolant having a phase transition temperature

(75) Inventor: Mitchell Levinson, Pleasanton, CA (US)

Correspondence Address: JUNIPER MEDICAL, INC.;PERKINS COIE, LLP, P.O. BOX 1247, SEATTLE, WA, 98111-1247, US

(73) Assignee: Juniper Medical, Inc., Pleasanton, CA (US)

(21) Appl. No.: 11/435,502

(22) Filed: May 17, 2006

(51) Int. Cl. A61F 7/00

(52) U.S. Cl. 607/108

(57) ABSTRACT

Cooling devices, such as a thermally conductive device, systems, and methods for removing heat from subcutaneous lipid-rich cells, and more particularly to a coolant in a flexible membrane wherein the coolant preferably has a phase transition temperature less than or approximately equal to about 0° C. The thermally conductive device may have compartments with varying phase transition temperatures to provide differential cooling to a treatment region. Alternatively, the thermally conductive device has a stratification of layers with varying phase transition temperatures to provide an increasing or decreasing temperature profile. The thermally conductive device may further have an anatomically conformal shape. The thermally conductive device, for example, can be triangular shaped for an abdomen region, oval shaped for a hip region, figure eight shaped for a buttocks region, or rectangular shaped for a thigh region.

### TECHNICAL FIELD

The present application relates to cooling devices, systems, and methods for removing heat from subcutaneous lipid-rich cells, and more particularly to a coolant in a flexible membrane wherein the coolant has a phase transition temperature below 15° C., and preferably less than or equal to 0° C.

### BACKGROUND

Excess body fat increases the likelihood of developing various types of diseases such as heart disease, high blood pressure, osteoarthrosis, bronchitis, hypertension, diabetes, deep-vein thrombosis, pulmonary emboli, varicose veins, gallstones, hernias, and several other conditions.

In addition to being a serious health risk, excess body fat can also detract from personal appearance and athletic performance. For example, excess body fat can form cellulite that causes an “orange peel” effect at the surface of the skin. Cellulite forms when subcutaneous fat protrudes into the dermis and creates dimples where the skin is attached to underlying structural fibrous strands. Cellulite and excessive amounts of fat are often considered to be unappealing. Thus, in light of the serious health risks and aesthetic concerns associated with excess fat, an effective way of controlling excess accumulation of body fat is urgently needed.

Liposuction is a method for selectively removing body fat to sculpt a person's body. Liposuction is typically performed by plastic surgeons using specialized surgical equipment that mechanically removes subcutaneous fat cells via suction. One drawback of liposuction is that it is a serious surgical procedure, and the recovery may be painful. Liposuction can have serious and occasionally even fatal complications. In addition, the cost for liposuction is usually substantial.

Conventional non-invasive treatments for removing excess body fat typically include topical agents, weight-loss drugs, regular exercise, dieting, or a combination of these treatments. One drawback of these treatments is that they may not be effective or even possible under certain circumstances. For example, when a person is physically injured or ill, regular exercise may not be an option. Similarly, weight-loss drugs or topical agents are not an option when they cause an allergic or negative reaction. Furthermore, fat loss in selective areas of a person's body cannot be achieved using weight-loss drugs.

Other non-invasive treatment methods include applying heat to a zone of subcutaneous lipid-rich cells. U.S. Pat. No. 5,948,011 discloses altering subcutaneous body fat and/or collagen by heating the subcutaneous fat layer with radiant energy while cooling the surface of the skin. The applied heat denatures fibrous septa made of collagen tissue and may destroy fat cells below the skin, and the cooling protects the epidermis from thermal damage. This method is less invasive than liposuction, but it still can cause thermal damage to adjacent tissue.

Another promising method of reducing subcutaneous fat cells is to cool the target cells as disclosed in U.S. Patent Publication No. 2003/0220674, the entire disclosure of which is incorporated herein. This publication discloses, among other things, reducing the temperature of lipid-rich subcutaneous fat cells to selectively affect the fat cells without damaging the cells in the epidermis. Although this publication provides promising methods and devices, several improvements for enhancing the implementation of these methods and devices would be desirable including providing a portable, disposable device that is inexpensive to manufacture.

U.S. Patent Publication No. 2003/0220674 also discloses methods for selective removal of lipid-rich cells, and avoidance of damage to other structures including dermal and epidermal cells. A method for inducing collagen compaction, remodeling and formation is also needed for treatment of loose or sagging skin, age- or sun-damaged skin or a variety of other skin disorders. Therefore, a method for simultaneously removing lipid-rich cells while providing beneficial collagen effects is also needed.

### BRIEF DESCRIPTION OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 is a graph of internal energy versus temperature for an exemplary phase transition of a coolant in accordance with an embodiment of the invention.

FIG. 2 is a sectional view of a portion of a thermally conductive device having a stratification of layers with varying phase transition temperatures to provide an increasing or decreasing temperature profile for removing heat from subcutaneous lipid-rich cells in accordance with an embodiment of the invention.

FIG. 3 is a schematic view of a system for removing heat from subcutaneous lipid-rich cells of a subject in accordance with embodiments of the invention.

FIG. 4 is a cross section along lines 4-4 of FIG. 3 in accordance with an embodiment of the invention.

FIGS. 5A and 5B are sectional views of the thermally conductive device illustrating a thermally conductive device having a curved surface in accordance with another embodiment of the invention.

FIGS. 6A-6D are schematic views of thermally conductive devices illustrating exemplary shapes of the thermally conductive device in accordance with another embodiment of the invention.

FIG. 7 is a schematic view of a thermally conductive device having baffles or compartments to provide a multi-compartmental thermally conductive device in accordance with another embodiment of the invention.

### DETAILED DESCRIPTION

In the following description, certain specific details are set forth in order to provide a thorough understanding of various embodiments of the invention. However, one skilled in the relevant art will recognize that the invention may be practiced without one or more of these specific details, or with other methods, components, materials, etc. In other instances, well-known structures associated with the thermally conductive device have not been shown or described in detail to avoid unnecessarily obscuring descriptions of the embodiments of the invention.

Unless the context requires otherwise, throughout the specification and claims which follow, the word “comprise” and variations thereof, such as, “comprises” and “comprising” are to be construed in an open, inclusive sense that is as “including, but not limited to.”

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Further more, the particular features, structures, or characteristics may be combined in any suitable manner in one or more embodiments.

The headings provided herein are for convenience only and do not interpret the scope or meaning of the claimed invention.

### A. Overview

The present disclosure describes devices, systems, and methods for cooling subcutaneous lipid-rich cells. The term “subcutaneous tissue” means tissue lying underneath the dermis and includes adipocytes (fat cells) and subcutaneous fat. It will be appreciated that several of the details set forth below are provided to describe the following embodiments in a manner sufficient to enable a person skilled in the relevant art to make and use the disclosed embodiments. Several of the details and advantages described below, however, may not be necessary to practice certain embodiments of the invention. Additionally, the invention can include other embodiments that are within the scope of the claims but are not described in detail with respect to the Figures.

One aspect is directed toward a thermally conductive device for removing heat from subcutaneous lipid-rich cells, and more particularly to a coolant in a flexible membrane wherein the coolant has a phase transition temperature less than 15° C. The phase transition temperature is preferably less than or approximately equal to 0° C.,; however, any phase transition temperature which would effect the selective removal of lipid-rich cells and avoidance of damage to other structures, including non-lipid-rich cells, would be within the scope of the present invention. Another aspect is directed toward a thermally conductive device having compartments with varying phase transition temperatures to provide differential cooling to a treatment region. Another aspect is directed toward a thermally conductive device having a stratification of layers with varying phase transition temperatures to provide an increasing or decreasing temperature profile over time.

Another aspect is directed toward a thermally conductive device having an anatomically conformal shape. The thermally conductive device, for example, can be triangular shaped for an abdomen region, oval shaped for a hip region, figure eight shaped for a buttocks region, or rectangular shaped for a thigh region. Another aspect of the invention is directed toward a thermally conductive device capable of treating such that the tissue is in a folded configuration. In such an embodiment, the tissue is pulled up and away from the body so that it can be clamped or held between two cold surfaces, or one cold surface and an opposing surface. In this embodiment, tissue contact pressure may be controlled by clamping the tissue in the apparatus. In this embodiment, the thermally conductive device may be foldable so that it could be folded over the tissue.

Another aspect is directed toward a thermally conductive device having a thermal interface in thermal communication and configured to contact a subject's skin. The thermal interface can be capable of reducing a temperature of a region such that lipid-rich cells in the region are affected while non-lipid-rich cells are not generally affected. Further aspects include that the thermal interface can be a curved surface for concentrating the cooling effects.

Another aspect is directed toward a method of applying a thermally conductive device for a specified period of treatment time to reduce a temperature of a region such that lipid rich cells in the region are affected while non-lipid-rich cells are not generally affected. Further aspects include a method directed toward applying pressure during the treatment time to increase the effectiveness of the treatment.

### B. Phase Transition: Freezing

In physics, a phase transition, (or phase change) is the transformation of a thermodynamic system from one phase to another. The distinguishing characteristic of a phase transition is an abrupt sudden change in one or more physical properties, in particular the heat capacity, with a small change in a thermodynamic variable such as the temperature. The liquid to solid transition is called the freezing phase transition.

In physics and chemistry, freezing is the process of cooling a liquid to the temperature (called freezing point) where it turns solid. Melting, the process of turning a solid to a liquid, is the opposite of freezing. For most coolants, melting and freezing temperatures are equal. However, rapid cooling by exposure to cryogenic temperatures can cause a coolant to freeze below its melting point, a process known as flash freezing.

Since the melting point and the freezing point of a fluid are the same, the temperature of a frozen mass will remain stable over a period of time to allow the fluid to either fully melt or fully freeze. At the phase transition, the melting point of the fluid is going from solid (e.g. ice) to liquid (e.g. water) as heat is added. Alternatively, the freezing point is going from liquid to solid as heat is taken away.

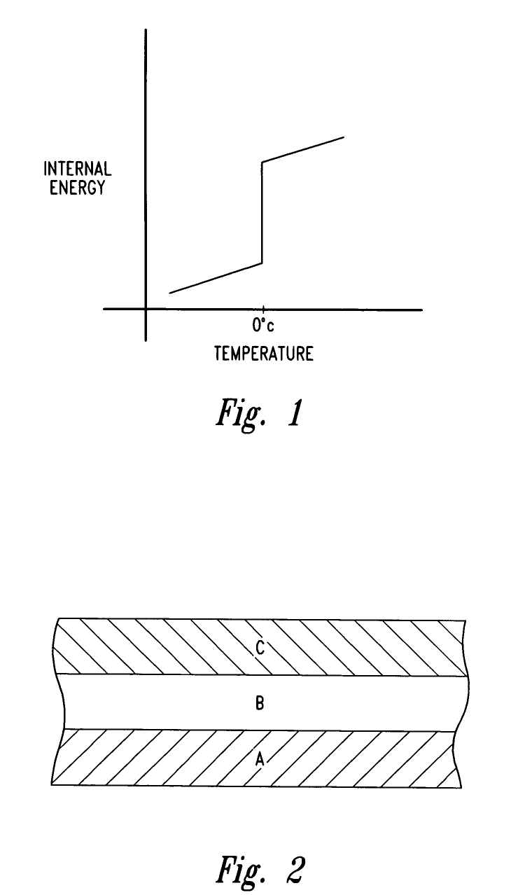

As illustrated in FIG. 1, the result of the melting temperature equaling the freezing temperature of any substance is that (every other condition being equal) the temperature at which the substance goes from a solid state to a liquid state is the same as the temperature at which this substance goes from a liquid state to a solid state, and thus the temperature will remain stable over a period of time while the fluid is fully transitioning phases. The result is that solid water (ice) at 0° C. and liquid water at 0° C. will coexist for a period of time. Over this time, the amount of solid water will decrease as the amount of liquid water increases and as the internal energy changes. The difference between the solid water and the liquid water (being that they are at the same temperature) is that the water molecules are organized differently in the solid water as it is in liquid water. In liquid water, the molecules of water are not localized to one spot, whereas in solid water, the molecules of water are kept in place. This means that to get liquid water to become organized into an ice form must require a release of energy. Conversely, the same amount of energy is released in order to make the ice become liquid water. Thus, since the melting point is the same as the freezing point, the change occurring is in the direction of the flow of energy as illustrated in FIG. 1.

Pure ice cannot super-heat: being even slightly above 0° C. forces it to promptly start melting, to an amount which exactly uses up the heat that has been added to it while it is at 0° C. This is referred to as the latent heat of fusion of melting/freezing. The presence of ice and water in contact with each other allows a gradual but barrier-free exchange in equilibrium to happen between ice and water, or between liquid and solid phases of almost any mixture. In accordance with the present invention, the energy removed from the skin of a subject at the interface between a thermally conductive device and the skin will be approximately equal to this latent heat of fusion. For water, for example, the latent heat of fusion is 80 Calories/gram. That is, if 80 Calories of energy are removed from the skin at the interface by the ice, then 1 gram of water is converted from ice to water.

For purposes of simplicity, ice/water is discussed as an exemplary fluid; however, water is not unique in this process. The melting point for any substance is the same as its freezing point and many mixtures may be used to yield a lower phase transition temperature. For example polypropylene glycol (PPG) added to water will reduce the phase transition temperature depending on the ratio of PPG to water. According to alternative aspects, mixtures of water, polypropylene glycol, glycerine, polyethylene glycol, alcohol, and/or similar substances will provide phase transition temperatures in the range of about −20° C. to about 0° C.

Another exemplary mixture is salt and water. A mixture of salt and water results in a phase transition temperature of less than 0° C., down to approximately −2° C. As the liquid water transitions to ice, the growing ice rejects the salt and contains only the water. Forcing the salt out of the water mixture costs energy, resulting in a freezing phase transition temperature of approximately −2° C. After a water ice-berg is formed in the salt water mixture, the puddle of fresh-water in the middle melts at 0° C. The result is a 2° C. difference between melting and freezing. The fresh-water mixes back into the salt water mixture, and the cycle is completed with an energy loss. Thus, the salt water mixture is a substance with unequal melting and freezing points.

According to aspects of the invention, the coolant or fluid in the thermally conductive device has a phase transition temperature equal to a target surface temperature at the skin interface. For example, the coolant may have a phase transition temperature of −3° C. and may have a thermal mass sized to hold a constant phase transition temperature for a time period in the range of 2 minutes to 60 minutes, more preferably for a time period in the range of 5 minutes to 40 minutes, and most preferably for a time period in the range of 10 minutes to 25 minutes. Alternatively, the coolant may have a phase transition temperature in the range of −20° C. to about 15° C., preferably a phase transition temperature in the range of −15° C. to about 5° C., and more preferably a phase transition temperature in the range of −10° C. to about 0° C., and most preferably a phase transition temperature in the range of −10° C. to about −2° C.

### C. Phase Transition Fluid

The fluid in phase transition may take the form of a solid fluid, slurry, supercooled fluid, frozen granules, or a combination thereof. Various forms of the fluid will be advantageous to specific embodiments as described further below. For example, a solid fluid may allow the thermally conductive device to retain a specific configuration for a period of time. According to aspects of the invention, the solid fluid is in a convex shape to allow the thermally conductive device to apply constant pressure or differential pressure to the skin interface. According to another aspect, the solid fluid is in a convex shape to allow the thermally conductive device to accommodate a body contour and provide constant pressure across the skin interface. According to still another aspect, the fluid may be frozen granules, supercooled fluid or slurry to allow the thermally conductive device to conform to a body contour and provide uniform cooling to the skin interface.

FIG. 2 illustrates a section view of a thermally conductive device having a stratification of layers with varying phase transition temperatures to provide an increasing or decreasing temperature profile over time. According to this aspect, a time-temperature profile is created by including different solids in series within the thermally conductive device wherein each solid had a different phase transition temperature. As shown in FIG. 2, a first solid A has a first phase transition temperature, a second solid B has a second phase transition temperature, and a third solid C has a third phase transition temperature. In operation, as one solid finished melting or transitioning, the next solid enters into its phase transition and maintains, increases, or decreases the temperature of an exterior of the thermally conductive device. According to alternative embodiments of the invention, the thermally conductive device may include one or a plurality of solids having the same or different phase transition temperatures.

### D. System for Selectively Reducing Lipid-rich Cells: Flexible Thermally Conductive Device

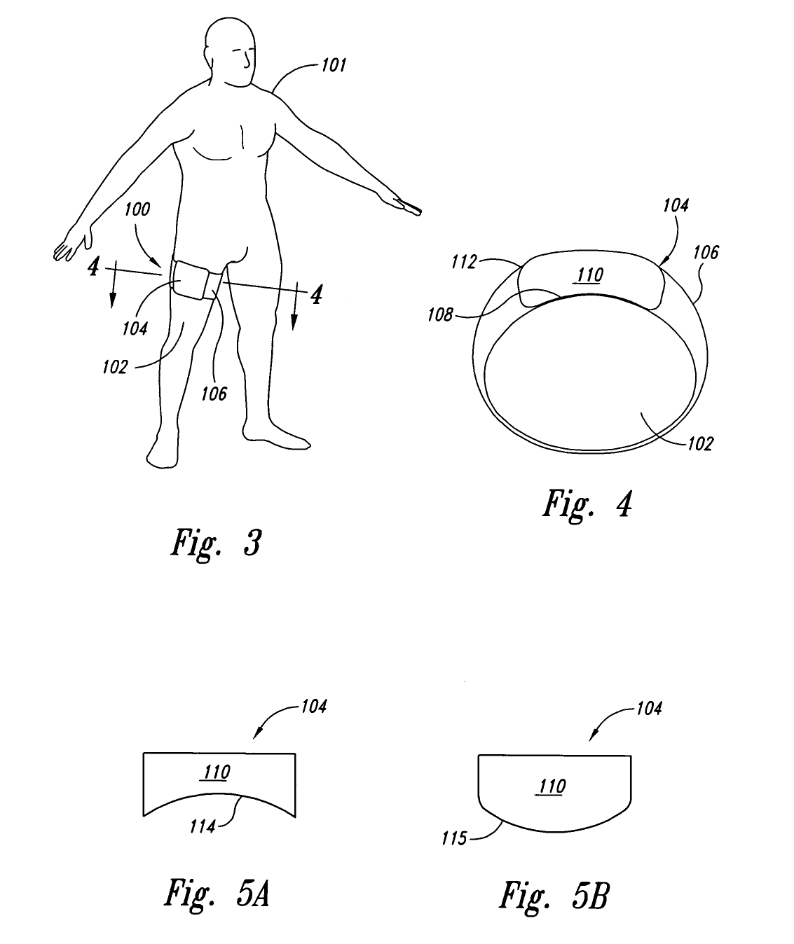

FIG. 3 illustrates a thermally conductive device 104 and, for purposes of illustration, is shown attached to a subject 101 for a cooling treatment. FIG. 3 is a schematic view of a system 100 for removing heat from subcutaneous lipid-rich cells of a subject 101. The system 100 can include a thermally conductive device 104 placed at a thigh area 102 of the subject 101 or another suitable area for removing heat from the subcutaneous lipid-rich cells of the subject 101. The thermally conductive device 104 includes a coolant contained in a flexible membrane. The thermally conductive device 104 may further include an elastomeric band or other retention device 106 for holding the thermally conductive device in place during treatment. The retention device 106 may be integral to the thermally conductive device 104 or may affix or retain the thermally conductive device 104 separately. For example, a separate retention device may be an elastic bandage wrap as is common in the medical device industry. The retention device 106 may further apply pressure to the thermally conductive device in a treatment region to increase the effectiveness of the treatment. Various embodiments of the thermally conductive device 104 are described in more detail below with reference to FIGS. 4-7.

FIG. 4 is a cross section along lines 4-4 of FIG. 3. The thermally conductive device 104 includes a phase transition temperature coolant 110 contained in a flexible membrane 112. The flexible membrane 112 may be cellophane-type material or a polyester film such as Mylar®, or any other thermally conductive, thin and/or flexible material. The membrane 112 may directly contact the skin at the skin interface 108 or a coupling fluid (not shown) may be placed between the skin interface 108 and the membrane 112. The membrane 112 is chosen to provide a minimal thermal loss or thermal gradient between the phase transition temperature of the coolant 110 and the skin of the subject 101. In accordance with aspects of the invention, the flexible membrane 112 of the thermally conductive device 104 readily conforms to the contours of the subject.

Alternatively, the thermally conductive device 104 may include a semi-rigid or rigid membrane 114 having a curved surface, shown as a concave surface in FIG. 5A. In operation, a curved surface may serve to distribute the cooling effect in a treatment region. According to yet another embodiment of the invention, the thermally conductive device 104 includes a semi-rigid or rigid membrane 115 having a convex surface as shown in FIG. 5B. A convex surface may apply pressure and concentrate the cooling effect to a treatment region. In operation, distributing the cooling effect and/or applying increased pressure increases the effectiveness of the cooling treatment in the treatment region.

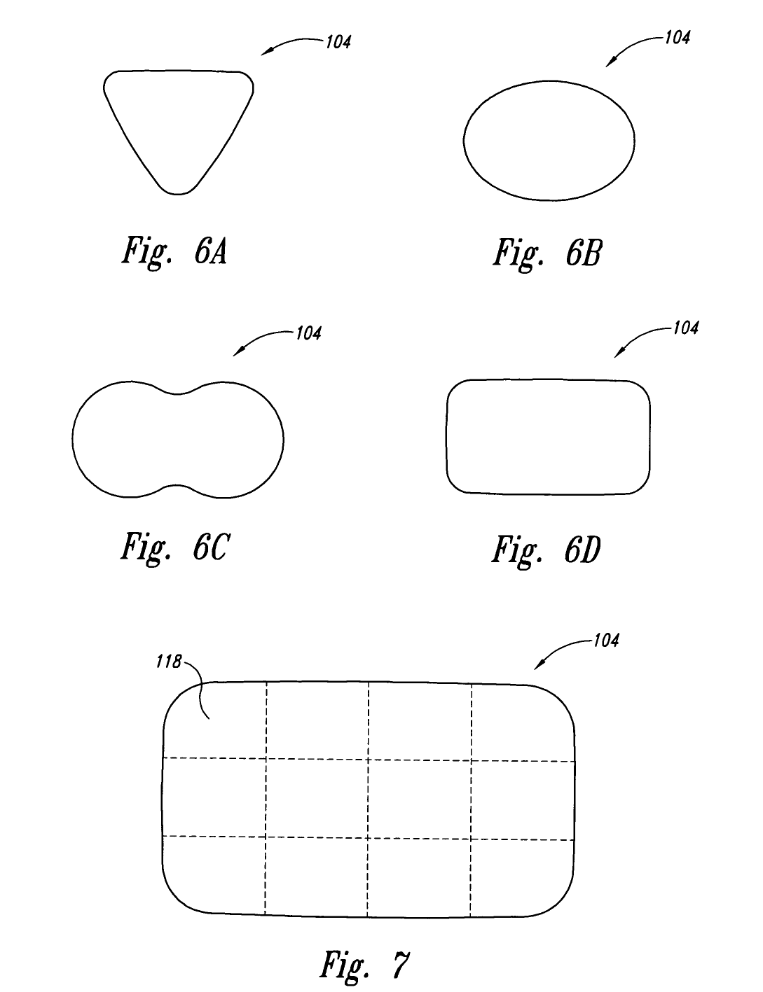

According to further aspects, the thermally conductive device 104 is configured in a specific shape to provide an anatomically conformal shape. The thermally conductive device as shown in FIGS. 6A-D, for example, can be triangular shaped for an abdomen region as shown in FIG. 6A; an oval shaped for a hip region as shown in FIG. 6B; a figure eight shaped for a buttocks region as shown in FIG. 6C; or rectangular shaped for a thigh region as shown in FIG. 6D. Alternatively, the thermally conductive device 104 may be of any conceivable shape and size to facilitate treatment to the treatment region.

One expected advantage of the portable system 100 is that the cooling device 104 can be applied to the subject 101 irrespective of the current physical condition of the subject 101. For example, the system 100 can be applied even when the subject 101 is not ambulatory or is ill. Another expected advantage is that the system 100 can remove or affect fat non-invasively without piercing the skin of the subject 101. Yet another expected advantage is that the system 100 is compact and can be used in an outpatient facility or a doctor's office.

### E. Thermally Conductive Device Configuration

Another aspect is directed toward a thermally conductive device having compartments with varying phase transition temperatures to provide differential cooling to a treatment region. Alternatively, the thermally conductive device has compartments to provide flexibility and to distribute the coolant across the thermally conductive device. Thus, FIG. 7 is an alternative example of the thermally conductive device 104 in accordance with one example of the invention for use in the system 100. This alternative example, and those alternative examples and other alternatives described herein, are substantially similar to previously-described examples, and common acts and structures are identified by the same reference numbers. Only significant differences in operation and structure are described with respect to FIG. 7. In this example, the thermally conductive device 104 includes baffles or compartments 118 to provide a multi-compartmental thermally conductive device. According to aspects of the invention, the compartments 118 may be fluidicly interconnected or may be distinct compartments containing coolants of varying phase transition temperatures.

The thermally conductive device can have many additional embodiments with different and/or additional features without detracting from the operation the device. For example, the thermally conductive device may or may not have multiple compartments. The coolant in a first compartment can have a phase transition temperature lower than a coolant in a second compartment to provide differential cooling. The thermally conductive device may be in a specific shape.

One expected advantage of using the thermally conductive device 104 is that subcutaneous lipid-rich cells can be reduced generally without collateral damage to non-lipid-rich cells in the same region. In general, lipid-rich cells can be affected at low temperatures that do not affect non-lipid-rich cells. As a result, lipid-rich cells, such as those forming the cellulite, can be affected while other cells in the same region are generally not damaged even though the non-lipid-rich cells at the surface are subject to even lower temperatures. Another expected advantage of the thermally conductive device 104 is that it is relatively compact because the thermally conductive device 104 can be configured in any size and shape. Yet another advantage is that the thermally conductive device can be applied to various regions of the subject's body because the thermally conductive device can be sized and shaped to conform to any body contour. Another expected advantage is that by pressing the thermally conductive device 104 against the subject's skin, blood flow through the treatment region can be reduced to achieve efficient cooling.

### F. Method of Applying a Thermally Conductive Device

Another aspect is directed toward a method applying a thermally conductive device for a specified period of treatment time to reduce a temperature of a region such that lipid rich cells in the region are affected while non-lipid-rich cells are not generally affected. Further aspects include a method directed toward applying pressure during the treatment time to increase the effectiveness of the treatment.

Applying the thermally conductive device to provide pressure to the subject's skin or pressing against the skin can be advantageous to achieve efficient cooling. In general, the subject 101 has a body temperature of about 37° C., and the blood circulation is one mechanism for maintaining a constant body temperature. As a result, blood flow through the dermis and subcutaneous layer of the region acts as a heat source that counteracts the cooling of the sub-dermal fat. As such, if the blood flow is not reduced, cooling the subcutaneous tissues would require not only removing the specific heat of the tissues but also that of the blood circulating through the tissues. Thus, reducing or eliminating blood flow through the target region can improve the efficiency of cooling and avoid excessive heat loss from the dermis and epidermis.

By cooling the subcutaneous tissues to a temperature lower than 37° C., subcutaneous lipid-rich cells can be selectively affected. In general, the epidermis and dermis of the subject 101 have lower amounts of unsaturated fatty acids compared to the underlying lipid-rich cells forming the subcutaneous tissues. Because non-lipid-rich cells usually can withstand colder temperatures better than lipid-rich cells, the subcutaneous lipid-rich cells can be selectively affected while maintaining the non-lipid-rich cells in the dermis and epidermis. An exemplary range for the coolant may include a phase transition temperature in the range of −20° C. to about 15° C., preferably a phase transition temperature in the range of −15° C. to about 5° C., and more preferably a phase transition temperature in the range of −10° C. to about 0° C., and most preferably a phase transition temperature in the range of −10° C. to about −2° C. The lipid-rich cells can be affected by disrupting, shrinking, disabling, destroying, removing, killing, or otherwise being altered. Without being bound by theory, selectively affecting lipid-rich cells is believed to result from localized crystallization of highly saturated fatty acids at temperatures that do not induce crystallization in non-lipid-rich cells. The crystals can rupture the bi-layer membrane of lipid-rich cells to selectively necrose these cells. Thus, damage of non-lipid-rich cells, such as dermal cells, can be avoided at temperatures that induce crystal formation in lipid-rich cells. Cooling is also believed to induce lipolysis (e.g., fat metabolism) of lipid-rich cells to further enhance the reduction in subcutaneous lipid-rich cells. Lipolysis may be enhanced by local cold exposure, inducing stimulation of the sympathetic nervous system.

In certain embodiments, once a desired temperature is achieved, the temperature of the region can be maintained for a pre-determined period of time. The cooling cycle can be terminated by removing the thermally conductive device from the skin or by designing the phase transition temperature to completely transition after a predetermined period of time. After a certain period of time, a thermally conductive device 104 having a specific phase transition temperature can be reapplied to the same portion of the skin as described above until a desired reduction in lipid-rich cells is achieved. In another embodiment, a thermally conductive device 104 of specific phase transition temperature can be applied to a different portion of the skin as described above to selectively affect lipid-rich cells in a different subcutaneous target region.

One expected advantage of several of the embodiments described above is that the thermally conductive device 104 can selectively reduce subcutaneous lipid-rich cells without unacceptably affecting the dermis, epidermis and/or other tissues. Another expected advantage is that the thermally conductive device 104 can simultaneously selectively reduce subcutaneous lipid-rich cells while providing beneficial effects to the dermis and/or epidermis. These effects may include: fibroplasias, neocollagenesis, collagen contraction, collagen compaction, collagen density increase, collagen remodeling, and acanthosis (epidermal thickening). Another expected advantage is that the thermally conductive device 104 can conform to various body contours of a subject. Furthermore, another expected advantage is that the system 100 is portable, compact and efficient such that the method described above can be administered in an outpatient clinic, doctor's office, or patient's home instead of in a hospital.

The above description of illustrated embodiments, including what is described in the Abstract, is not intended to be exhaustive or to limit the invention to the precise forms disclosed. Although specific embodiments of and examples are described herein for illustrative purposes, various equivalent modifications can be made without departing from the spirit and scope of the invention, as will be recognized by those skilled in the relevant art. The teachings provided herein of the invention can be applied to thermally conductive devices, not necessarily the exemplary thermally conductive devices generally described above.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the invention can be modified, if necessary, to employ coolants with various phase transition temperatures, thermally conductive devices with various configurations, and concepts of the various patents, applications and publications to provide yet further embodiments of the invention.

These and other changes can be made to the invention in light of the above-detailed description. In general, in the following claims, the terms used should not be construed to limit the invention to the specific embodiments disclosed in the specification and the claims, but should be construed to include all phase transition liquids and devices that operated in accordance with the claims. Accordingly, the invention is not limited by the disclosure, but instead its scope is to be determined entirely by the following claims.

From the foregoing, it will be appreciated that specific embodiments of the invention have been described herein for purposes of illustration, but that various modifications may be made without deviating from the spirit and scope of the invention. Accordingly, the invention is not limited except as by the appended claims.

I/We claim:

1. A cooling device for cooling subcutaneous lipid rich cells in a region of a subject having skin, comprising:
a thermally conductive device having a flexible membrane configured to interface with a subject's skin at a treatment region; and
a coolant encapsulated in the flexible membrane, the coolant having a defined phase transition at relatively constant temperature over a period of time wherein the thermally conductive device is configured to reduce a temperature of the region such that lipid rich cells in the region are affected while non-lipid rich cells in the epidermis are not generally affected.

2. The cooling device of claim 1 wherein the phase transition temperature is less than or approximately equal to 0° C.

3. The cooling device of claim 1, further comprises multiple coolants having differing phase transition temperatures to provide an increasing and/or decreasing temperature profile over time for removing heat from subcutaneous lipid-rich cells.

4. The cooling device of claim 3 wherein the coolants are in a solid state and each coolant forms a layer such that the encapsulated coolant is stratified in layers of differing phase transition temperatures.

5. The cooling device of claim 1 wherein the membrane is a thermally conductive polymer film.

6. The cooling device of claim 1 wherein the thermally conductive device is generally in the shape of one of: triangular shaped, oval shaped, figure eight shaped and rectangular.

7. The cooling device of claim 1 further comprising a plurality of compartments defined by the membrane of the thermally conductive device.

8. The cooling device of claim 7 wherein the plurality of compartments are fluidicly connected.

9. The cooling device of claim 1, wherein the thermally conductive device is configured for reducing the temperature of the surface of the region to a range of about −15° C. to about 5° C.

10. The cooling device of claim 1, wherein the phase transition temperature is less than about −2° C.

11. A method of applying a thermally conductive device including a coolant having a phase transition temperature encapsulated in a membrane, comprising:
(i) reducing the temperature of the coolant in the thermally conductive device to or below a solid-fluid transition temperature;
(ii) applying the thermally conductive device to a subject's skin such that the membrane encapsulating the coolant interfaces with the subject's skin; and
(iii) transitioning the coolant through a phase transition temperature to cool the region under the thermally conductive device to a temperature that disrupts lipid rich cells in the region without generally disrupting non-lipid rich cells in the epidermis.

12. The method of claim 11, further comprising
terminating cooling the region by removing the thermally conductive device from the region; and
repeating steps (i)-(iii).

13. The method of claim 11, further comprising
removing the thermally conductive device from the region after disrupting lipid rich cells of the region; and
reducing lipid rich cells of a different region by performing stages (i)-(iii) as applied to the different region of the skin.

14. The method of claim 11, further comprising maintaining the fluid at a relatively constant temperature for a predetermined treatment period.

15. A system for cooling a region of skin of a subject, comprising:
a conformal thermally conductive device having a coolant encapsulated in a flexible membrane, the coolant having a phase transition temperature, the membrane being configured for reducing a temperature of the region of skin of a subject such that lipid rich cells in the region are affected while non-lipid rich cells are not affected; and
a retaining element for holding the thermally conductive device in place during a treatment period.

16. The system of claim 15 wherein the retaining element is one of an elastomeric wrap, an adhesive and an adjustable strap having a fastening element.

17. The system of claim 15 wherein the coolant has a phase transition temperature less than or approximately equal to about 0° C.

18. The system of claim 15 wherein the phase transition temperature is constant for a treatment period in the range of about 5 minutes to about 60 minutes.

19. The system of claim 15 wherein the coolant includes water, glycol, glycerin, polypropylene glycol, alcohol and/or salt.

20. The system of claim 15, further comprises multiple coolants having differing phase transition temperatures to provide an increasing and/or decreasing temperature profile over time for removing heat from subcutaneous lipid-rich cells.

21. The system of claim 15, further comprising a thermally conductive coupling fluid positioned between the subject's skin and the flexible membrane.
